(19) United States
(12) Patent Application Publication
(10) Pub. No.: US 2021/0363173 A1
(43) Pub. Date: Nov. 25, 2021

(54) NEUROSTERIOD DERIVATIVES AND USES THEREOF

(71) Applicant: ACERUS BIOPHARMA INC., Mississauga (CA)

(72) Inventors: Nathan Bryson, Toronto (CA); Avinash C. Sharma, Brampton (CA)

(21) Appl. No.: 17/392,004

(22) Filed: Aug. 2, 2021

Related U.S. Application Data
(63) Continuation of application No. 16/403,100, filed on May 3, 2019, now abandoned.
(60) Provisional application No. 62/667,100, filed on May 4, 2018.

(51) Int. Cl. C07J 7/00; A61K 47/14; A61K 47/22; A61K 47/44

(52) U.S. Cl. CPC C07J 7/009; A61K 47/44; A61K 47/22; A61K 47/14

(57) ABSTRACT

The present invention contemplates novel neurosteroid derivative compounds, such as derivatives of ganaxolone and allopregnanolone, having improved solubility and bioavailability. The novel neurosteroid derivative compounds are characterized by the following formulas:

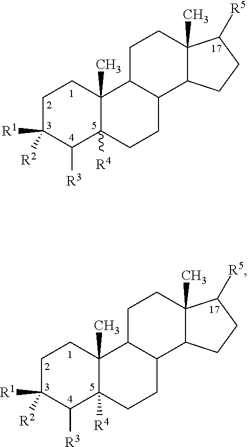

wherein R¹ is methyl or hydrogen, R² is an ester function (R—C(O)O—), R³ is hydrogen, R⁴ is alpha or beta hydrogen, R⁵ is R—CO— or any hydrocarbon structure (R—), and wherein R (in R² or R⁵) is independently selected from any structure comprising 10 carbon atoms or fewer, which is linear or branched, saturated or unsaturated, may comprise cyclic or aromatic functions within the structure, and wherein R contains no more than 1 OH or NR₂, or 2 ether or thioether functions.

### CROSS-REFERENCES TO RELATED APPLICATIONS

This application is a continuation application of U.S. patent application Ser. No. 16/403,100, filed on May 3, 2019 which claims priority to and the benefit of U.S. Provisional Application No. 62/667,100, filed on May 4, 2018 both of which are hereby incorporated by reference in their entirety as if fully set forth herein.

### FIELD OF THE INVENTION

The present invention relates to new neurosteroid derivative compounds having improved solubility and bioavailability, and to pharmaceutical compositions comprising, as an active ingredient, the novel neurosteroid derivative compounds, and novel pharmaceutical compositions formulated with a neurosteroid derivative compounds, articles of manufacture of pharmaceutical preparations formulated with a neurosteroid derivative compounds, and therapeutic uses thereof for treating medical conditions, such as pain, e.g., acute, and/or neuropathic pain and fibromyalgia, mood disorders, e.g., depression, major depression, postpartum depression, bipolar, anxiety, and movement disorders, e.g., epilepsy, tremors and Parkinson's Disease, and for improving therapeutic effects and outcomes.

### BACKGROUND OF THE INVENTION

Neurosteroids are steroid hormone derivatives that are devoid of hormonal action but still affect neuronal excitability through modulation of ionotropic receptors. Neurosteroids are further subdivided into natural (produced in the brain) and synthetic. Some authors distinguish between hormonal and regular neurosteroids in the group of natural ones. The latter group, including hormone metabolites like allopregnanolone or tetrahydrodeoxycorticosterone, is devoid of hormonal activity. Both hormones and their derivatives share, however, most of the physiological functions. It is usually very difficult to distinguish the effects of hormones and their metabolites. All these substances may influence their activity in regulation of the pro- and anti-apoptotic factors expression, intracellular signaling pathways, neurotransmission, oxidative, and inflammatory processes. Multiple studies have been conducted so far to show efficacy of neurosteroids in the treatment of the central and peripheral nervous system injury, ischemia, neurodegenerative diseases, or seizures.

Allopregnanolone, also known as 5α-pregnan-3α-ol-20-one, is an endogenous inhibitory pregnane neurosteroid. While it is a potent positive allosteric modulator of the action of γ-aminobutyric acid (GABA) at GABAA receptor, it has low solubility in aqueous-based liquids. Allopregnanolone has effects similar to those of other positive allosteric modulators of the GABA action at GABAA receptor such as the benzodiazepines, including anxiolytic, sedative, and anticonvulsant activity. Endogenously produced allopregnanolone exerts a pivotal neurophysiological role by fine-tuning of GABAA receptor and modulating the action of several positive allosteric modulators and agonists at GABAA receptor. See Borowitz, et al. Front. Endocrin. 2011, 2, 1.

Ganaxolone, 3a-hydroxy-3-methyl-5a-pregnan-20-one or (3a, 5a)-3-hydroxy-3-methylpregnan-20-one or CCD 1042, is a synthetic neurosteroid analogue that acts as a modulator of GABA receptors. Ganaxolone has been tested for safety in clinical trials, and has relatively modest side effects even at very high doses. It has shown promise for treating temporal lobe seizures, as well as catamenial epilepsy. Ganaxolone is also under study for the treatment of post-traumatic stress disorder, Fragile-X syndrome, neuropathic pain, neonatal seizures and post-partum depression. Ganaxolone is a neurosteroid taught to be a possible anticonvulsant and antiepileptic with potential utility in the treatment of generalized absence seizures as well as simple and complex partial seizures. See Carter, et al.: J. Pharm. And Exp. Ther., Vol. 280, #3, 1284-1295. Ganaxolone is also taught to be a positive allosteric modulator of GABAA, but failed to show benefit on time to pain relief in a phase 2 clinical trial for migraine. Ganaxolone has been approved by the U.S. FDA for the treatment of protocadherin-19 gene (PCDH19) female epilepsy. Ganaxolone is well-tolerated in adults and children.

GABAA receptors mediate a significant portion of the first inhibitory synaptic transmission in the central nervous system. In addition to neurosteroids, such as ganaxolone, a number of compounds such as benzodiazepines, barbiturates and general anesthetics also bind with distinct sites in the GABAA receptor protein thereby acting as potent allosteric modulators of the receptor. Among the benzodiazepines (e.g., valium) and barbiturates (e.g., phenobarbital), there are well known antiepileptics that have been used to treat a variety of seizures in the clinic. These compounds have demonstrated a significant efficacy in a variety of preclinical animal models of seizure activity. In addition, they are also known to be potent anxiolytics, muscle relaxants and sedatives. To this date, there is no documented evidence that these allosteric modulators of the GABAA receptor protein have significant efficacy in pain models, both acute and neuropathic pain conditions.

Unfortunately, Ganaxolone also has limited solubility in aqueous-based liquids. As such, typical aqueous-based liquid pharmaceutical preparations with >1% concentration of Ganaxolone are generally formulated as suspensions or dispersions of solids. Pharmaceutical liquids comprising ganaxolone have been described in U.S. Publication No. 20130287851, published on Oct. 31, 2013. The low solubility of Ganaxolone may be at least partially responsible for its low bioavailability in-vivo, as it is reported that nearly 80% of the drug is recovered in feces post-oral administration.

Thus, there is a definite need for improving solubility and bioavaiability of ganaxolone and allopregnanolone. There is a need for improved formulations capable of delivering Ganaxolone and or allpregnanolone in a more efficient manner for improving their therapeutic effects and outcomes.

U.S. Publication No. 20030211162, published on Nov. 13, 2003, describes a method of spray drying solutions of ganaxolone to produce small particles to enhance the rate of solubilisation and enhance effectiveness. Such particles are stabilized for use as powders for solid dosage forms and as dispersions for liquid dosage forms, as per U.S. Publication Nos. 20070148252, published on Jun. 28, 2007 and 20070141161, published on Jun. 21, 2007. Also, esters of alpha- and beta-forms of Ganaxolone have been described for the treatment of neuropathic pain were described in U.S. Patent Publication No. 20060009432, published on Jan. 12, 2006 although examples of these new compositions, their solubility and usages are lacking.

Thus, there are definite needs for novel neurosteroidal-type compounds with improved solubility and pharmaceutical compositions formulated with same having improved bioavailability for treating various medical conditions, such as, pain, e.g., acute and neuropathic pain and fibromyalgia, mood disorders, e.g., depression, major depression, postpartum depression, bipolar, anxiety, and movement disorders, e.g., epilepsy, seizures, tremors and Parkinson's Disease, for improving therapeutic effects and outcomes.

### SUMMARY OF THE INVENTION

The present invention overcomes the above-mentioned problems and drawbacks of the present state of the art with respect to neurosteroids through the discovery of novel neurosteroid derivatives, pharmaceutical compositions formulated with same and methods of their use.

Generally speaking, the present invention provides for modified neurosteroids with pharmaceutically cleavable ester functions, wherein, the novel neurosteroids are characterized by formula (I), as follows:

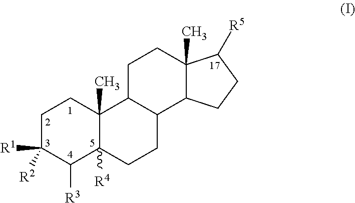

wherein R¹ is methyl or hydrogen, R² is an ester function (R—C(O)O—), R³ is hydrogen, R⁴ is alpha or beta hydrogen, R⁵ is R—CO—, or any hydrocarbon structure (R—), and wherein R (in R² or R⁵) is independently selected from any structure comprising 10 carbon atoms or fewer, which is linear or branched, saturated or unsaturated, may comprise cyclic or aromatic functions within the structure, and wherein R contains no more than 1 OH or NR₂, or 2 ether or thioether functions.

Specifically speaking, R⁴ is preferably in the alpha-position to provide novel modified neurosteroid compounds characterized by formula (II):

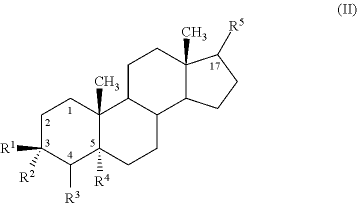

wherein R¹ is methyl or hydrogen, R² is an ester function (R—C(O)O—), R³ is hydrogen, R⁴ is alpha or beta hydrogen, R⁵ is R—CO— or any hydrocarbon structure (R—), and wherein R (in R² or R⁵) is independently selected from any structure comprising 10 carbon atoms or fewer, which is linear or branched, saturated or unsaturated, may comprise cyclic or aromatic functions within the structure, and wherein R contains no more than 1 OH or NR₂, or 2 ether or thioether functions.

In accordance with the present invention, it is directed to a novel pharmaceutical composition comprised of:

(a) a modified neurosteroid compound which is characterized by formula (I) or formula (II), and (b) a pharmaceutically acceptable excipient, wherein the novel pharmaceutical compositions are suitable for treating a medical condition, for example, acute and/or neuropathic pain and fibromyalgia, mood disorders (depression, major depression, postpartum depression, bipolar, anxiety) or movement disorders (epilepsy, tremors, Parkinson's Disease).

The present invention is also directed to an article of manufacture exemplified by a composition comprising: (a) a modified neurosteroid compound which is characterized by formula (I) or formula (II), and (b) at least 1 pharmaceutically acceptable excipient, and (c) a label with instructions for using the composition to treat a medical condition, such as, acute and/or neuropathic pain and fibromyalgia, movement disorders, such as epilepsy, seizures, tremors, and Parkinson's Disease, or mood disorders, such as depression.

The present invention is further directed to a novel method for preparing a modified neurosteroid, as characterized above under Formula I or Formula II, pharmaceutical composition useful for treating such medical conditions, which method comprises (a) combining a modified neurosteroid, as characterized above under Formula I or Formula II, with a pharmaceutically acceptable excipient to form a novel pharmaceutical formulation acceptable for administration to a subject, e.g., an animal including a human; and (b) packaging the formulation with written instructions for the treatment of a medical condition, such as, acute and/or neuropathic pain and fibromyalgia, movement disorders, such as epilepsy, seizures, tremors, and Parkinson's Disease, or mood disorders, by administering the novel pharmaceutical formulation to a patient in need of such treatment at a prescribed effective amount in accordance with a prescribed treatment regimen.

Still further, the present invention is drawn to a method for treating a medical condition, such as acute and/or neuropathic pain, which method comprises: Administration of a therapeutic dose of the neurosteroid composition to a patient in need thereof.

It should be further understood that the above summary of the present invention is not intended to describe each disclosed embodiment or every implementation of the present invention. The description further exemplifies illustrative embodiments. In several places throughout the specification, guidance is provided through examples, which examples can be used in various combinations. In each instance, the examples serve only as representative groups and should not be interpreted as exclusive examples.

Thus, the following detailed description and examples are provided to aid the understanding of the present invention. It therefore should be understood that any modifications can be made in, e.g., the formulations, methods and procedures set forth without departing from the invention.

### DETAILED DESCRIPTION AND PREFERRED EMBODIMENTS

In accordance with the present invention and as used herein, the following terms are defined with the following meaning, unless explicitly stated otherwise.

The term “physiologically cleavable ester” refers to a derivative of the hydroxyl of the neurosteroid of formula (I) and an acid or acid derivative, wherein the product is cleaved in the body to give the compound formula (I) or an active metabolite.

Such a physiologically cleavable ester can be viewed as a “pro-drug.” Such a “pro-drug” is particularly valuable if it increases the bioavailability of the corresponding hydroxyl compound (where R2 is hydroxyl) when such a pro-drug is administered to a subject. For example, a “pro-drug” administered orally may be more readily absorbed into the blood, may facilitate the delivery of the parent compound to a biological compartment of the subject such as tissue, cells, tumors, molecular targets and organs, like the brain or lymphatic system, may allow for the development of alternative pharmaceutical preparations such as oral solids or enteral medications (capsules, gel capsules, tablets, orally-disintigratable tablets, sublingual tablets, caplets, pills, lozenge, troches, powders, liquids, solutions, suspensions, elixirs, emulsions, syrups, tinctures, etc.), transdermals, including topicals, vaginal or suppositories (creams, gels, ointments, lotions, foams, transdermal patches, sprays, roll-ons, waxes, capsules, ovules, inhalants, etc.), nasal/oral (aqueous gels, oleaginous gels, sprays, aerosols, inhalants, etc.), rectal and vaginal (enemas, suppositories, douches, etc.) and injectable (epidural, intravenous, intramuscular, subcutaneous, intradermal, intracardiac, intraocular, intrathecal, intra-articular, intramuscular bolus, etc.) which may also have more favorable patient acceptance, safety profiles and/or pharmacokinetics for specific tailoring to patients for use in the intended indication.

A general overview of pro-drugs is provided, (1) “Pro-drugs As Novel Delivery Systems,” Vol. 14 of the ACS Symposium Series, by T. Higuchi and V. Stella, and (2) “Bioreversible Carriers in Drug Design,” American Pharmaceutical Association, Porgamon Press, 1987, Edward B. Roche, Ed.

Carboxylic acids that form the “carbonoyl group” R that can be used as derivatives according to the present invention and form the “pro-drug” group R2 include mono-carboxylic acids that are derived from unsubstituted or substituted lower linear or branched chain alkyl, alkenyl, alkynyl or arylakyl entities. Naturally occurring carboxylic acids are generally a preferred class of that may as acceptable, cleavable esters of a pharmaceutically-active ingredient.

The term “lower alkyl” carboxylic acid refers to a monovalent, saturated aliphatic hydrocarbon radical having from one to twelve (12) carbon atoms bonded to a carboxyl group. Alkyl may be a straight chain (i.e. linear), a branched chain, or a cyclic structure. Representative examples of lower alkyl radicals include methyl, ethyl, n-propyl, n-butyl, n-pentyl, n-hexyl, isopropyl, isobutyl, isopentyl, amyl, sec-butyl, tert-butyl, tert-pentyl, cyclopropyl, cyclobutyl, cyclopentylethyl (cypionate), undecanoate and the like.

The radical may be optionally substituted with substituents at positions that do not significantly interfere with the preparation of compounds falling within the scope of the present invention and that do not significantly reduce the efficacy of the novel compounds. The alkyl may be optionally substituted with one to three substituents independently selected from the group consisting of halo, hydroxyl, ether, cyano, nitro or amino.

The term lower “alkenyl” carboxylic acid refers to an aliphatic group that has 1-12 carbons, may be straight chain, branched chain, and cyclic groups and with no more than 3 double bonds, all of which may be optionally substituted similarly to the alkyl group. Representative examples of lower alkenyl radicals in carboxylic acids include vinyl (ethenyl), allyl (propen-3-yl), 1-buten-4-yl; 2-buten-4-yl, 1-penten-5-yl, and the like.

The term lower “alkynyl” carboxylic acid refers to unsaturated hydrocarbon groups which contain at least one carbon-carbon triple bond and includes straight chain and branched chain groups which may be optionally substituted. Suitable alkynyl groups include propyn-3-yl, pentyn-5-yl, and the like which may be optionally substituted similarly to the alkyl group.

Aromatic carboxylic acids are those carboxylic acids characterized by the presence of at least one benzene ring or an entity that resembles benzene. Thus, without limitation, aromatic carboxylic acids include benzoic acid, 2-phenylethanoic acid, ortho-, meta- and para-methylbenzoic acid. Aromatic carboxylic acids may also be substituted with a substituent that does not significantly reduce the efficacy, e.g., one to five lower alkyls, halo, hydroxyl, nitro, lower alkoxy, amino, cyano, and the like.

Further, substituted carboxylic acid may contain a non-carbon atom such as N, S, or 0 linked to the carbon chain of the fatty acid carboxyl group. Thus, the hetero carboxylic acid is (R)2-N—R′—C(O)OH, RS—R′—C(O)OH, or R—(O—R′)n-C(O)OH, wherein R is defined as previously (vide infra) and R′ being an branched or straight chain alkyl, lower alkenyl, or lower alkynl, alkylaryl or arylalkyl group, optionally with heteroatom substitutions and having a molecular weight of no greater than 200 g/mol.

The term “neuroactive steroid” refers to an endogenous steroid (or its synthetic analog) that rapidly alters the excitability of neurons by direct action on membrane ion channels, including GABA-A and NMDA receptors.

The term “pharmaceutically-acceptable carboxylic acid” means a carboxylic acid moiety that is useful for forming the pharmaceutical formulations and compositions, are physiologically acceptable and generally non-toxic to a subject receiving the moiety.

Pharmaceutical Compositions

Methods of nasal administration of hormone-based drugs are known, for example, an oil based vehicle for drug administration is described in U.S. Pat. No. 8,877,230, issued on Nov. 4, 2014, and U.S. Published Application No. 20120009250 published Jan. 12, 2012 entitled “Controlled Release Delivery System for Nasal Applications”.

Neuropeptides in general, and ganaxolone and allopregnanolone more specifically, are poorly soluble in most common water-based pharmaceutical vehicles, as indicated above, and are also poorly soluble in vegetable oils which can also be used for administering drugs in liquid form. This is a result of a structure that is dominated by multiple rings of hydrocarbons, mostly devoid of polar groups (see core structure of neurosteroids in (FIG. 1)).

Making esters of neurosteroids may increase the solubility of the parent drug in oil based vehicles. Vegetable oils can present different solubility of steroid compounds. See Riffkin et al.: J Pharm Sci 1964, 53(8), 891.

Some examples of lipid-based vehicles for oral delivery have been described in U.S. Pat. No. 6,096,338, issued on Aug. 1, 2000. Silica similar to this has been used to make thixotropic compositions since the 1980's and is described in U.S. Pat. No. 4,497,918, issued on Feb. 5, 1985. Thixotropic macroemulsions comprising both oil and aqueous phases useful in the present invention are described in U.S. Publication No. 20170348276, published on Dec. 7, 2017.

According to certain embodiments in accordance with the present invention, the formulation comprises: (1) an esterified ganaxalone derivative; (2) an oily vehicle; and (3) a wetting agent or mixture of wetting agents and/or a pharmaceutically acceptable surfactant or mixture of surfactants.

According to certain embodiments in accordance with the present invention, the formulation comprises: (1) an esterified ganaxalone derivative; (2) an oily vehicle; (3) a wetting agent or mixture of wetting agents and/or a pharmaceutically acceptable surfactant or mixture of surfactants; and (4) a thickening agent.

According to certain embodiments in accordance with the present invention, the formulation comprises: (1) an esterified ganaxalone derivative; (2) an oily vehicle; (3) a wetting agent or mixture of wetting agents and/or a pharmaceutically acceptable surfactant or mixture of surfactants; (4) a thickening agent; and (5) optionally water.

According to certain embodiments in accordance with the present invention, the formulation comprises: (1) an esterified ganaxalone derivative; (2) an oily vehicle; (3) a wetting agent or mixture of wetting agents and/or a pharmaceutically acceptable surfactant or mixture of surfactants; (4) a thickening agent; and optionally water, wherein the combination of ingredients forms a thixotropic mixture.

According to certain embodiments in accordance with the present invention, the formulation comprises: (1) an esterified ganaxalone derivative; (2) an oily vehicle; (3) a wetting agent or mixture of wetting agents and/or a pharmaceutically acceptable surfactant or mixture of surfactants; and (4) a thickening agent, such as colloidal silica, wherein the combination of ingredients forms a thixotropic mixture.

According to certain embodiments in accordance with the present invention, representative examples of an oily vehicle include a pharmaceutically acceptable vegetable oil, a monoglyceride, a diglyceride, benzyl benzoate, sucrose acetate isobutyrate (SAIB), a synthetic triglyceride, a synthetic oil, and any combination or mixtures thereof.

According to certain embodiments in accordance with the present invention, representative examples of a pharmaceutically acceptable vegetable oil include Almond Oil Sweet (Prunus dulcis), Almond Oil Virgin (Prunus amygdalus), Aloe Vera Oil (Aloe barbadensis), Apricot Kernel Oil (Prunus armeniaca), Argan Oil (Argania spinosa), Avocada Oil (Persea americana), Apricot Oil (Prunus armeniaca), Amla Oil (Emblica officinalis), Borage Oil (Borago officinalis), Black Seed Oil (Nigella sativa), Carrot Oil (Daucus carota), Coconut Oil (Cocus nucifera), Corn Oil, Cucumber Oil (Cucumis sativa), Chaulmogra Oil (Hydnocarpus wightianus), Emu Oil (Dromaius novae-Hollandiae), Evening Primrose Oil (Oenothera biennis), Flaxseed Oil (Linum usitatissimum), Grapeseed Oil (Vitus vinifera), Hazel Nut Oil (Avekkana), Jojoba Oil Refined (Simmondsia chinensis), Moringa Oil (Moringa oliefera), Marula Oils (Sclerocarya birrea), Wheatgerm Oil, Triticum vulgare, Macadamia Oil, (Macadamia ternifolia), Musk Melon Oil (Cuvumis melon), Musk Oil (Abelmoschus moschatus), Mustered Oil, Neem Oil (Azadirachta indica), Olive Oil (Olea europaea), Peach Kernel Oil (Prunus persica), Peanut Oil (Arachis hypogeae), Pomegranate Oil, Punica granatum, Psoralea Oil (Psoralea corylifolia), Primrose Oil (Oenothera bienni), Papaya Seed Oil (Carica papaya), Rosehip Seed Oil (Rosa rubiginosa), Safflower Oil, Seasame Seed (Refined) (Sesamum indicum), Sea Buckthorn Oil (Hippophae rhamnoides), Soya Bean Oil (Soja hispida), Sunflower Oil (Helianthus annus), Sweet Almond Oil (Prunus amygdalus Var. Dulcus), Sweet Cherry Kernel Oil (Prunus avium), Walnut Oil (Juglans regia), Water Melon Oil (Citrullus vulgaris).

Pharmaceutically acceptable synthetic oils according to the present invention include SAIB, polyethylene glycol (PEG), polyethyleneglycol-polypropylene glycol (poloxamers), alkyl-modified PEG or poloxamers, silicone and mineral oil

According to certain preferred embodiments in accordance with the present invention, the oily vehicles include medium chain triglycerides, castor oil, sesame oil, PEG, Poloxamer, SAIB or mixtures thereof.

According to certain embodiments in accordance with the present invention include the ganaxolone therapeutic active or mixture of actives includes one or more compounds described by formula 1.

According to certain embodiments in accordance with the present invention, the ganaxolone therapeutic active or mixture of actives includes one or more compounds described by formula 2.

According to certain embodiments in accordance with the present invention, the ganaxolone therapeutic active is pure or a mixture of actives, resulting from one or more forms of the alpha forms of the isomers of the compounds in Formula 2.

According to certain embodiments in accordance with the present invention, the ganaxolone therapeutic active includes ganaxolone proprionate, ganaxolone enanthate, ganaxolone cypionate, ganaxolone undecanoate, and combinations or mixtures thereof.

According to certain embodiments, a wetting agent or mixture of wetting agents and/or a pharmaceutically acceptable surfactant or mixture of surfactants includes a polysorbate, a polyoxyethylene hydrogenated vegetable oil, a polyoxyethylene vegetable oil, a polyoxyethylene sorbitan fatty acid ester, a polyoxyethylene-polyoxypropylene block copolymer, a polyglycerol fatty acid ester, a polyoxyethylene glyceride, a polyoxyethylene sterol, or a derivative or analogue thereof, a reaction mixture of polyols and at least one member of the group consisting of fatty acids, glycerides, vegetable oils, hydrogenated vegetable oils, fractionated oils and sterols, a tocopheryl polyethylene glycol succinate, a sugar ester, a sugar ether, a sucroglyceride, an alkylglucoside, an alkylmaltoside, an alkylthioglucosides, a lauryl macrogolglyceride, a polyoxyethylene alkyl ether, a polyoxyethylene alkylphenol, a polyethylene glycol fatty acid ester, a polyethylene glycol glycerol fatty acid ester, a polyoxyethylene sorbitan fatty acid ester, a polyoxyethylene-polyoxypropylene block copolymer such as poloxamer-108, 188, 217, 238, 288, 338, 407, 124, 182, 183, 212, 331, or 335 or combinations thereof, an ionic hydrophilic surfactant such as sodium dodecyl sulphate or docusate sodium, a bile acid, a cholic acid, a deoxycholic acid, a chenodeoxycholic acid, and salts thereof, and mixtures thereof.

According to certain embodiments of the present invention, the formulation further comprises a rheology modifying (thickening agent) agent. The thickening agent would preferably be added to the majority liquid phase (oil or water) of the formulation. For formulations where the majority phase is an oil (with or without an aqueous phase), the pharmaceutically acceptable thickening agents include colloidal silica, silicates, alumina, a high molecular weight polymer or a solid/waxy substance, bee wax, alumina, silica, colloidal silica, silicates and high melting waxes, and/or cetostearyl alcohol. For formulations where the majority phase is aqueous, the thickener is preferably a pharmaceutically acceptable hydrophilic polymer such as HPMC, HPC, Sodium CMC, Sodium CMC and MCC, natural gums like Xanthan gum, Guar gum, gum acacia, gum tragacanth, starches like maize starch, potato starch, and pregelatinized starch. Thickening agents may be added to both phases in a mixed phase system.

According to certain embodiments of the present invention, a formulation comprising water may further comprise a surfactant and an osmotic complement.

Also according to certain embodiments of the present invention, examples of surfactants include Glycol Distearate, Sorbitan Trioleate, Propylene Glycol Isostearate, Glycol Stearate, Sorbitan Sesquioleate, Lecithin, Sorbitan Oleate, Sorbitan Monostearate NF, Sorbitan Stearate, Sorbitan Isostearate, Steareth-2, Oleth-2, Glyceryl Laurate, Ceteth-2, PEG-30 Dipolyhydroxystearate, Glyceryl Stearate SE, Sorbitan Stearate (and) Sucrose Cocoate, PEG-4 Dilaurate, Methyl Glucose Sesquistearate, Lecithin HLB (variable) PEG-8 Dioleate, Sorbitan Laurate, Sorbitan Laurate, PEG-40 Sorbitan Peroleate, Labrafil M1944CS, Laureth-4, PEG-7 Glyceryl Cocoate, PEG-20 Almond Glycerides, PEG-25 Hydrogenated Castor Oil, Stearamide MEA, Glyceryl Stearate (and) PEG-100 Stearate, Polysorbate 85, PEG-7 Olivate, Cetearyl Glucoside, Stearamide MEA, PEG-8 Oleate, Polyglyceryl-3 Methyglucose Distearate, Oleth-10, Oleth-10/Polyoxyl 10 Oleyl Ether NF, Ceteth-10, PEG-8 Laurate, Cocamide MEA, Polysorbate 60 NF, Polysorbate 60, Polysorbate 80, Isosteareth-20, PEG-60 Almond Glycerides, PEG-20 Methyl Glucose Sesquistearate, Ceteareth-20, Oleth-20, Steareth-20, Steareth-20, Steareth-21, Steareth-21, Ceteth-20, and Steareth-100.

According to certain preferred embodiments in accordance with the present invention, preferably, the neurosteroid therapeutic active is an enanthate ester of the active steroid, the oily vehicle is castor oil, and the wetting agent is oleoyl polyoxylglycerides. Optionally, silica maybe used as the preferred thickener.

Compounds useful in the present invention are those of formula (I), as defined herein. Ganaxolone (3a-hydroxy-3b-methyl-5a-prenan-20-one) and Allopregnanolone (3a-hydroxy-5a-prenan-20-one) are preferred compounds. As mentioned here-in-before, a physiologically cleavable ester of the 3-hydroxy group, especially of ganaxolone, is also useful. While the carboxylic acids from which such esters may be derived were generically mentioned previously, the following is a list of carboxylic acids useful to form the esters at the 3-position: acetic acid, n-propionic acid, n-butyric acid, t-butyl carboxylic acid, n-pentanoic acid, benzoic acid, morpholinocarboxylic acid, malonic acid, succinic acid, glutaric acid, adipic acid, pimelic acid, suberic acid, n-propenoic acid, e-butenoic acid, and the like. Esters derivatives of neurosteroids may be found by referring to U.S. Pat. No. 5,939,545, issued on Aug. 17, 1999.

Compositions of the invention may be administered by any suitable route which will introduce the intended compound to the patient in a soluble form and therefore overcome the solubility limitation of the parent active compounds. The compositions of the present invention are prodrugs and after administration, via the action of hydrolases or natural hydrolysis, are converted to the parent active compound. The mode of administration may be orally (including buccally or sublingual), parenterally (e.g., intravenously, intramuscularly, subcutaneously, subdermally) topically (transdermally) or any other acceptable route other than through the intestine), by suppository (vaginally or anally), and other routes that may be apparent to one of skill in the art and as described in paragraph 30 here-in-above.

Pharmaceutically acceptable excipients include solvents, diluents, binders, lubricants, preservatives, disintegrants, wetting agents, surfactants, stabilizers, anti-oxidants, coloring agents, flavors, sweeteners, and the like. Examples of these excipients can be found in the standard publication Remington's Pharmaceutical Sciences, 19 Edition, Mack Publishing Co., Easton, Pa.-1995 (“Remington's”). Techniques for preparing formulations will be found in detail in Remington's.

Dosage forms according to the present invention include liquids, oils, semi-solid emulsions or creams, solids, waxes, capsules and tablets, as well as those listed in paragraph 30 here-in-above, which can be administered to a patient. The preferred route of administration is one that provides the drug to the patient in an efficient and convenient manner while achieving the safety and efficacy for the desired condition.

The preferred dosage of a chosen drug will depend upon both the potency of the drug and the status of the patient. The composition will need to be prescribed by a treating physician, who will take into account any relevant factors, such as the age and weight of the patient, the severity of the patient's symptoms, and the chosen route of administration.

Depending on the dosage form and the administration route, the amount of the active compound in the composition to be administered will be sufficient to deliver the desired amount of active to the subject being treated to alleviate, modulate or prevent the medical condition, i.e., a therapeutically effective amount. Thus another aspect of the present invention is the use of a component of formula (I) to prepare a composition useful for the treatment of a medical condition. The compound is confined with an excipient to form an acceptable formulation then combined with a label providing written instructions for administration.

Another aspect of the present invention is a pharmaceutical composition suitable for treating a medical condition, which composition comprises a compound of formula (I) and a pharmaceutically-acceptable excipient. Generally the amount of the active compound will vary from about 1 milligram (mg) to about 500 mg per dosage unit, preferably about 2 mg-100 mg, and most preferably about 5 mg-50 mg. Depending on the size of the dosage form, the active may vary between about 1% to about 90% by weight, preferably less than 50% by weight.

Thus, the percentage of the active may be, e.g., 1, 2, 3, 4, 5, 10, 20, 30, 40, 50 percent or any intermediate percentage or range as desired. By using a dosage form with the desired composition percentage, a doctor skilled in the art can administer enough to achieve about 0.1 mg/kilogram (kg) body weight in the subject to about 100 mg/kg, prefer-ably about 0.1 mg/kg to about 10 mg/kg. The label that accompanies the dosage form will provide instructions for using the composition to treat the medical condition. Treatment can be on an as-needed, acute, subchronic (for a short period of time) or on a chronic basis.

Compositions may include a combination of different ester pro-drug actives at all ratios, up to the limit of solubility of each of the prodrugs in the composition, such that the combinations achieves a higher concentration of the parent active molecule in the composition than can be achieved with any of the component prodrugs in the composition.

Compositions of the invention can further be combined with other active ingredients.

Compositions according to the invention may be used to treat a number of medical, including neurological conditions. The preferred dose and route of administration may depend on the nature of the condition to be treated. Conditions that may be treated with neurosteroids according to the invention may include indications relating to (i) mood disorders, such as depression, major depression, postpartum depression, bipolar depression, anxiety, (ii) pain (acute, chronic, neuropathic, nociceptive, fibromyalgia, etc.) or (iii) movement disorders, such as various forms of seizure, epilepsy, Parkinsons disease and tremors.

All publications, patents, patent applications, etc. mentioned in the above specification are incorporated herein by reference in their entireties.

Various modifications and variations of the present invention will be apparent to those skilled in the art without departing from the scope and spirit of the invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

The following examples are provided as a guide for a practitioner of ordinary skill in the art. The examples should not be construed as limiting the invention, as the examples merely provide specific methodology useful in understanding and practicing an embodiment of the invention.

### EXAMPLES

### Example 1. Synthesis of Esters

Ganaxolone is reacted with about 1.25 equivalents of an acid chloride (propyl, heptanoyl (“enanthate”) or cyclohexylpropyl (“cypionate”)) in about 50 ml methylene chloride with gentle heating (about 60° C.) for about 1 h. The mixture is then extracted twice with about 200 ml about 0.1M aqueous phase. The organic phase is dried over Na2HCO3 and then was evaporated. The residue is purified by chromoatography until pure (about >98%) when controlled by HPLC.

Allopregnanalone esters are prepared in a similar manner.

### Example 2. Solubility of Esters in Oils

About 150 mg samples of each drug substance in Table are placed in about 300 mg of a test solvent in a test tube. The samples are swirled and gently heated with a warm water bath. Additional solvent is added in about 100 mg increments until solubility is reached. The samples are cooled overnight and if precipitation is observed, then more solvent is again added in portions until a room temperature solubility is obtained. The results are shown in Tables 1, 2 and 3. This results show the improved solubility of the ester forms in castor oil. Ganaxolone enanthate is a liquid and appeared miscible in the oils tested. This example shows the poor solubility of the parent steroids, and the significant increase in solubility of the actives when esterified.

TABLE 1
Solubility of different neurosteroid compounds in castor oil

| Drug substance | Solubility Limit |
|---|---|
| Allopregnanolone | about <10 mg/ml (<1% w/w) |
| Ganaxalone | about 10 mg/mL (1% w/w) |
| Ganaxalone Cypionate | about 150 mg/mL (15% w/w) |
| Ganaxalone Propionate | about 150 mg/mL (15% w/w) |
| Ganaxalone Enanthate | about >500 mg/mL (>50% w/w) |

TABLE 2
Solubility of Ganaxolone-cypionate in other solvents

| Solvent | Solubility Limit |
|---|---|
| Castor oil | about 150 mg/ml (15% w/w) |
| Sesame oil | about >272 mg/mL (>27.2% w/w) |
| Polyoxylglyceride-PEG6 esters | about >100 mg/ml (<10% w/w) |

TABLE 3
Solubility of Ganaxolone-propionate in other solvents

| Solvent | Solubility Limit |
|---|---|
| MCT | about >155 mg/mL (>15.5% w/w) |

### Example 3. 14% Ganaxolone Cypionate in a Castor Oil Formulation

Castor oil (about 82 parts) and oleoyl polyoxylglycerides (about 4 parts) is mixed together thoroughly. Ganaxalone cypionate (about 14 parts) is added and is dissolved upon mixing to form a clear gel or a viscous solution. The equivalent concentration of ganaxolone active is about 10.2%.

### Example 4. 15% Ganaxolone Propionate in a Castor Oil Formulation

Castor oil (about 81 parts) and oleoyl polyoxylglycerides (about 4 parts) are mixed together thoroughly. Ganaxalone propionate (about 15 parts) is added and is dissolved upon mixing to form a clear gel or a viscous solution. The equivalent concentration of ganaxolone active is about 12.9%.

### Example 5. 30% Ganaxolone Enanthate in a Castor Oil Formulation

Castor oil (about 66 parts) and oleoyl polyoxylglycerides (about 4 parts) is mixed together thoroughly. Ganaxalone enanthate (about 30 parts) is added and is dissolved upon mixing to form a clear gel or a viscous solution. The equivalent concentration of ganaxolone active is about 22.5%.

### Example 6. 36% Ganaxolone Enanthate in a Castor Oil Formulation

Castor oil (60 parts) and oleoyl polyoxylglycerides (about 3.7 parts) is mixed together thoroughly. Ganaxalone enanthate (about 36.3 parts) is added and dissolved upon mixing to form a clear gel or a viscous solution. The equivalent concentration of ganaxolone active is about 27.2%.

### Example 7. 36% Ganaxolone Enanthate in a Castor Oil Formulation

Colloidal silicon dioxide (about 4 parts) is dispersed in castor oil (about 92 parts). The oleoyl polyoxylglycerides (about 4 parts) are added and is mixed to form a uniform gel. The about 63.7 parts of this mixture is warmed to about 40° C. and is dissolved ganaxalone enanthate (about 36.3 parts) to form a clear gel. The equivalent concentration of ganaxolone active is about 27.2%.

### Example 8. Ganaxolone Ester Mixture in Castor Oil Formulation

Colloidal silicon dioxide (about 4 parts) is dispersed in castor oil (about 62.2 parts). The mixture is warmed to about about 40° C. and ganaxalone cypionate (4.9 parts), ganaxalone propionate (about 4.9 parts) and ganaxalone enanthate (about 20 parts) are added and are mixed to form a clear solution. Oleoyl polyoxylglycerides (4 parts) are added and are mixed to produce a uniform gel. The equivalent concentration of ganaxolone active is about 22.7%.

### Example 9. 36% Ganaxolone Enanthate Formulation

Colloidal silicon dioxide (about 4 parts) is dispersed in medium chain triglycerides (about 55.7 parts). The mixture is warmed to about about 40° C. and then ganaxalone enanthate (about 36.3 parts) is dissolved to form a clear mixture. Oleoyl polyoxylglycerides (about 4 parts) is then added and is mixed to form a uniform clear gel. The equivalent concentration of ganaxolone active is about 27.2%.

### Example 10. 36% Ganaxolone Enanthate Formulation

Colloidal silicon dioxide (about 4 parts) is dispersed in sesame oil (about 55.7 parts). The mixture is warmed to about about 40° C. and the ganaxalone enanthate (about 36.3 parts) is added to dissolve and form a clear mixture. Oleoyl polyoxylglycerides (about 4 parts) is added and is mixed to form a uniform clear gel. The equivalent concentration of ganaxolone active is about 22.7%.

### Example 11. 36% Ganaxolone Enanthate Formulation

Colloidal silicon dioxide (about 4 parts) is dispersed into a mixture of sesame oil (about 15.7 parts), medium chain triglycerides (about 20 parts) and castor oil (about 20 parts). The mixture is warmed to about about 40° C. and then ganaxalone enanthate (about 36.3 parts) is added to dissolve and form a clear mixture. Oleoyl polyoxylglycerides (about 4 parts) is added and is mixed to form a uniform clear gel. The equivalent concentration of ganaxolone active is about 27.2%.

### Example 12. Ganaxolone Ester Mixture Formulation

Colloidal silicon dioxide (about 4 parts) is dispersed into a mixture of sesame oil (about 20 parts), medium chain triglycerides (21.2 parts) and castor oil (about 21 parts). The mixture is warmed to about about 40° C. and then ganaxalone enanthate (about 20 parts), ganaxolone propionate (about 4.9 parts) and ganaxolone cypionate (about 4.9 parts) are dissolved to form a clear mixture. Oleoyl polyoxylglycerides (about 4 parts) is added and is mixed to form a uniform clear gel. The equivalent concentration of ganaxolone active is about 23%.

### Example 13. Ganaxolone Ester Mixture Formulation

To about 28 parts castor oil warmed to about about 40° C. is added ganaxolone propionate (about 3 parts) and ganaxolone cypionate (about 4 parts) and the mixture is stirred until all is dissolved forming a clear mixture. The esters comprised about 20% of the mixture. The equivalent concentration of the parent ganaxolone active is about 15.8%.

### Example 14. Ganaxolone Ester Mixture Formulation

Ganaxalone enanthate (about 81 parts), ganaxalone cypionate (about 16 parts) were dissolved in sesame oil (about 45 parts). The concentration of ganaxolone esters is about 68%. The equivalent concentration of parent ganaxolone active is about 50.1%.

### Example 15. Ganaxolone Ester Mixture Formulation

Ganaxalone enanthate (about 81 parts), ganaxalone cypionate (about 16 parts) is dissolved in castor oil (about 45 parts) to which silica (about 6 parts) and Oleoyl polyoxylglycerides (about 6 parts) are added with high shear mixing to form a gel. The concentration of ganaxolone esters is about 63%. The equivalent concentration of parent ganaxolone active is about 46%.

### Example 16. Emulsion Formulation of Ganaxolone Esters

Medium chain triglycerides (about 30 parts) and polyoxyl 35 castor oil (about 2 parts) is mixed. The mixture is heated to about 60° C. and then ganaxalone enanthate (about 36.3 parts) is added and is dissolved to form a clear oily solution. Separately, Carbomer 971P (about 0.4 parts) is dispersed in water (about 31.3 parts) at about 65° C. The oil phase-drug solution is added to the water phase-solution and is emulsified. The pH is adjusted with about 1N NaOH solution to about 6.5 to about 7.5. The mixture is cooled to about room temperature with continuous mixing to form a white opaque aqueous gel. The equivalent concentration of ganaxolone active is about 27.2%.

### Example 17. Gel Capsule Containing Ganaxolone

Neurosteroid composition (about 138 mg) from Example 10 (about 36.3% neurosteroid ester composition) is warmed with about 2 mg beeswax and is then cooled to about 40 C. The waxy product is poured into gelatin capsule while still warm and liquid, and is then weighed. The capsule is sealed to make a pharmaceutical dosage form providing about 50 mg of ganaxolone ester (equivalent to a dose of about 37 mg ganaxolone).

### Example 18. Nasal Dispenser Containing Ganaxolone

Neurosteroid composition from Example 7 (about 36.3% neurosteroid ester) is filled into a nasal dispenser as described in U.S. patent application Ser. No. 15/613,116 and then capped and is sealed. The nasal dispenser provides about 125 uL doses at each actuation. Use in the nose, this pharmaceutical dosage form can provide about 44.5 mg of ganaxolone ester per actuation (equivalent to a dose of about 33.3 mg ganaxolone) when it is administered to one nostril. When applied in both nostrils, the total dose administered is about 89 mg of ganaxolone esters (equivalent to a dose of about 66.6 mg ganaxolone).

### Example 19. Ganaxolone Parent Formulation

Colloidal silicon dioxide (about 4 parts) is dispersed into a mixture of sesame oil (about 15.7 parts), medium chain triglycerides (about 20 parts) and castor oil (about 20 parts). The mixture is warmed to about about 40° C. and then ganaxalone enanthate (about 0.5 parts) is added to dissolve and form a clear mixture. Oleoyl polyoxylglycerides (about 4 parts) is added and is mixed to form a uniform clear gel. The concentration of ganaxolone active is about 0.8%.

### Example 20. Ganaxolone Cypionate/Cannabidiol Gel Mixture

To castor oil (about 72 parts) that is warmed to about about 40° C. is added ganaxolone cypionate (about 10 parts) and cannabidiol (about 10 parts) and the mixture is stirred until about dissolved, giving a clear mixture. Colloidal silica (about 4 parts) and oleoyl polyoxylglycerides (about 4 parts) are added and are mixed using high shear to provide a uniform clear gel.

### Example 21. 25% Allopregnanolone Enanthate in a Castor Oil Formulation

Castor oil (about 71 parts) and oleoyl polyoxylglycerides (about 4 parts) are mixed together thoroughly. Allopregnanolone enanthate (about 25 parts) is added and is dissolved upon mixing to form a clear gel or a viscous solution. The equivalent concentration of allopregnanolone active is about 22%.

The complete disclosures of all publications cited herein are incorporated herein by reference in their entireties as if each were individually set forth in full herein and incorporated.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A neurosteroid compound having a formula comprising: 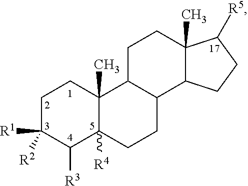
wherein R¹ is methyl or hydrogen, R² is an ester function (R—C(O)O—), R³ is hydrogen, R⁴ is alpha or beta hydrogen, R⁵ is R—CO— or any hydrocarbon structure (R—), and wherein R (in R² or R⁵) is independently selected from any structure comprising 10 carbon atoms or fewer, which is linear or branched, saturated or unsaturated, may comprise cyclic or aromatic functions within the structure, and wherein R contains no more than 1 OH or NR₂, or 2 ether or thioether functions.

2. The neurosteroid compound of claim 1, wherein R1 is methyl and R2 is physiologically cleavable ester and R4 is in the alpha configuration.

3. The neurosteroid compound of claim 1, wherein R1 is hydrogen and R2 is physiologically cleavable ester and R4 is in the alpha configuration.

4. A pharmaceutical composition comprising (a) a compound or mixture of physiologically cleavable ester compounds of claim 1, and (b) a pharmaceutically acceptable oily vehicle.

5. The pharmaceutical composition claim 4, wherein the pharmaceutically acceptable oily vehicle is a mixture of a vegetable oil and sucrose acetate isobutyrate (SAIB).

6. The pharmaceutical composition of claim 4, wherein the pharmaceutically acceptable oily vehicle is a vegetable oil or mixture of vegetable oils.

7. The pharmaceutical composition of claim 4, wherein the pharmaceutically acceptable oily vehicle is comprised of about greater than 75% castor oil.

8. The pharmaceutical composition of claim 4, wherein the pharmaceutically acceptable oily vehicle is comprised of neurosteroid compound greater than 75% medium chain triglycerides (MCT).

9. The pharmaceutical composition of claim 4 wherein the pharmaceutically acceptable oily vehicle is SAIB.

10. A pharmaceutical composition of claim 4, wherein the compound or mixture of physiologically cleavable ester compounds of claim 1 comprise a neurosteroid compound in an amount of about 1% to about 75% by weight of the composition.

11. A pharmaceutical composition of claim 4, wherein said pharmaceutical composition further comprises a wetting agent.

12. A pharmaceutical composition of claim 4, wherein said pharmaceutical composition further comprises a thickener.

13. A pharmaceutical composition of claim 4, wherein said pharmaceutical composition further comprises water.

14. A pharmaceutical composition of claim 12, wherein said pharmaceutical composition is thixotropic.

15. A pharmaceutical composition of claim 12, wherein said pharmaceutical composition is a solid or wax.

16. A pharmaceutical composition of claim 4, wherein the pharmaceutical composition is placed in a medical device, such as a cartridge, syringe, spray bottle, patch or dispenser bottle, for administration to a subject in need thereof.

17. A pharmaceutical composition of claim 4, wherein the pharmaceutical composition is placed in a gel capsule for administration to a subject in need thereof.

18. A pharmaceutical composition of claim 4, wherein the pharmaceutical composition is a topical dosage form (cream, gel or patch) for administration to a subject in need thereof.

19. A pharmaceutical composition of claim 4, wherein the pharmaceutical composition is a suppository for administration to a subject in need thereof.

20. A pharmaceutical composition of claim 4, wherein the pharmaceutical composition is placed in a syringe for injection for administration to a subject in need thereof.

21. A pharmaceutical composition of claim 4, wherein the pharmaceutical composition is placed in a syringe for injection for administration to a subject in need thereof.

22. A pharmaceutical composition that provides a daily dose of up to about 5 to 500 mg per day of any one or mixture of compounds of claim 1.

23. A method of use wherein a product or composition from claim 1 comprises the treatment to a patient in need thereof to treat a mood disorder (any form of depression, major depression, postpartum depression, bipolar, anxiety), pain (neuropathic pain, fibromyalgia) or movement disorder (seizure, epilepsy, involuntary movement (Parkinson's), dyskinesia, tremor, RLS).

24. A method of use where in the treatment comprises combining a product or composition of claim 1 and another medication (more specifically a medication compatible with the oily vehicle).

25. A neurosteroid compound having a formula comprising: 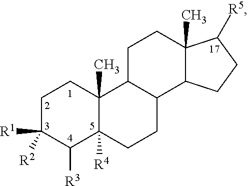
wherein R¹ is methyl or hydrogen, R² is an ester function (R—C(O)O—), R³ is hydrogen, R⁴ is alpha or beta hydrogen, R⁵ is R—CO— or any hydrocarbon structure (R—), and wherein R (in R² or R⁵) is independently selected from any structure comprising 10 carbon atoms or fewer, which is linear or branched, saturated or unsaturated, may comprise cyclic or aromatic functions within the structure, and wherein R contains no more than 1 OH or NR₂, or 2 ether or thioether functions.

26. A pharmaceutical composition comprising (a) a compound or mixture of physiologically cleavable ester compounds of claim 25, and (b) a pharmaceutically acceptable oily vehicle.

27. The pharmaceutical composition claim 25, wherein the pharmaceutically acceptable oily vehicle is a mixture of a vegetable oil and sucrose acetate isobutyrate (SAIB).

28. The pharmaceutical composition of claim 25, wherein the pharmaceutically acceptable oily vehicle is a vegetable oil or mixture of vegetable oils.

29. The pharmaceutical composition of claim 25, wherein the pharmaceutically acceptable oily vehicle is comprised of about greater than 75% castor oil.

30. The pharmaceutical composition of claim 25, wherein the pharmaceutically acceptable oily vehicle is comprised of neurosteroid compound greater than 75% medium chain triglycerides (MCT).

31. The pharmaceutical composition of claim 25 wherein the pharmaceutically acceptable oily vehicle is SAIB.

32. A pharmaceutical composition of claim 25, wherein the compound or mixture of physiologically cleavable ester compounds of claim 1 comprise a neurosteroid compound in an amount of about 1% to about 75% by weight of the composition.

33. A pharmaceutical composition of claim 25, wherein said pharmaceutical composition further comprises a wetting agent.

34. A pharmaceutical composition of claim 25, wherein said pharmaceutical composition further comprises a thickener.

35. A pharmaceutical composition of claim 25, wherein said pharmaceutical composition further comprises water.

36. A pharmaceutical composition of claim 34, wherein said pharmaceutical composition is thixotropic.

37. A pharmaceutical composition of claim 34, wherein said pharmaceutical composition is a solid or wax.

38. A pharmaceutical composition of claim 25, wherein the pharmaceutical composition is placed in a spray, inhalant or aerosol dispenser for administration to a subject in need thereof.

39. A pharmaceutical composition of claim 4, wherein the pharmaceutical composition is placed in a gel capsule for administration to a subject in need thereof.

40. A pharmaceutical composition of claim 4, wherein the pharmaceutical composition is a suppository for administration to a subject in need thereof.

41. A pharmaceutical composition that provides a daily dose of up to about 500 mg per day of any one or mixture of compounds of claim 25.

42. The neurosteroid compound of claim 25, wherein R1 is methyl and R2 is physiologically cleavable ester and R4 is in the alpha configuration.

43. The neurosteroid compound of claim 25, wherein R1 is hydrogen and R2 is physiologically cleavable ester and R4 is in the alpha configuration.

44. A pharmaceutical composition comprising: (1) an esterified ganaxalone derivative; (2) an oily vehicle; and (3) a wetting agent or mixture of wetting agents and/or a pharmaceutically acceptable surfactant or mixture of surfactants.

45. A pharmaceutical composition comprising: (1) an esterified ganaxalone derivative; (2) an oily vehicle; (3) a wetting agent or mixture of wetting agents and/or a pharmaceutically acceptable surfactant or mixture of surfactants; and (5) a thickening agent.

46. A pharmaceutical composition comprising: (1) an esterified ganaxalone derivative; (2) an oily vehicle; (3) a wetting agent or mixture of wetting agents and/or a pharmaceutically acceptable surfactant or mixture of surfactants; (4) a thickening agent; and (5) optionally water.

47. A thixotropic pharmaceutical composition comprising: (1) an esterified ganaxalone derivative; (2) an oily vehicle; (3) a wetting agent or mixture of wetting agents and/or a pharmaceutically acceptable surfactant or mixture of surfactants; (4) and a thickening agent; and (5) optionally water.

48. The thixotropic pharmaceutical composition of claim 47, wherein the thickening agent is colloidal silica.

49. A neurosteroid of claim 1, wherein the neurosteroid is selected from a group of neurosteroids consisting of ganaxolone proprionate, ganaxolone enanthate, ganaxolone cypionate, ganaxolone undecanoate, and combinations or mixtures thereof.

50. A neurosteroid of claim 25, wherein the neurosteroid is selected from a group of neurosteroids consisting of ganaxolone proprionate, ganaxolone enanthate, ganaxolone cypionate, ganaxolone undecanoate, and combinations or mixtures thereof.

51. A pharmaceutical composition for treating a subject, e.g., an animal or human, for a medical condition, wherein the subject is in need of treatment for the medical condition, said method composition comprising:
(a) a neurosteroid selected from a group of neurosteroids consisting of ganaxolone proprionate, ganaxolone enanthate, ganaxolone cypionate, ganaxolone undecanoate, and combinations or mixtures thereof, and
(b) a pharmaceutically acceptable excipient.

52. A method of treating a subject, namely, an animal or human, for a medical condition, wherein the subject is in need of treatment to treat the medical condition, said method comprises:
administering to the subject an effective amount of the pharmaceutical composition of claim 4 to treat the subject for the medical condition.

53. A method of claim 52, wherein the medical condition is pain.

54. A method of claim 53, wherein the pain is acute pain.

55. A method of claim 53, wherein the pain in neuropathic pain.

56. A method of claim 52, wherein the medical condition a mood disorder.

57. A method of claim 56, wherein the mood disorder is depression.

58. A method of claim 52, wherein the medical condition is a movement disorder.

59. A method of claim 58, wherein the movement disorder is selected from a group of mood disorders consisting of seizures, epilepsy, tremors and Parkinson's Disease.

60. A method of treating a subject, namely, an animal or human, for a medical condition, wherein the subject is in need of treatment to treat the medical condition, said method comprises:
administering to the subject an effective amount of the pharmaceutical composition of claim 25 to treat the subject for the medical condition.

61. A method of claim 60, wherein the medical condition is pain.

62. A method of claim 61, wherein the pain is acute pain.

63. A method of claim 61, wherein the pain is neuropathic pain.

64. A method of claim 60, wherein the medical condition a mood disorder.

65. A method of claim 64, wherein the mood disorder is depression.

66. A method of claim 60, wherein the medical condition is a movement disorder.

67. A method of claim 66, wherein the movement disorder is selected from a group of movement disorders consisting of seizures, epilepsy, tremors and Parkinson's disease.
